(19) United States
(12) Patent Application Publication
(10) Pub. No.: US 2009/0124553 A1
(43) Pub. Date: May 14, 2009

(54) Suppression of inflammation associated with transplantation using an epsilon PKC inhibitor

(76) Inventors: Daria D. Mochly-Rosen, Menlo Park, CA (US); Tomoyoshi Koyanagi, Kobe (JP); Koichi Inagaki, Otsu (JP); Akifumi Ootani, Palo Alto, CA (US)

Correspondence Address: King & Spalding LLP, P.O. Box 889, Belmont, CA, 94002-0889, US

(21) Appl. No.: 12/151,244

(22) Filed: May 2, 2008

Related U.S. Application Data
(60) Provisional application No. 60/927,580, filed on May 4, 2007.

(51) Int. Cl. A61K 38/10; A61K 38/08; A61P 37/06

(52) U.S. Cl. 514/13; 514/16; 514/15

(57) ABSTRACT

The described compositions and methods relate to the suppression of inflammatory responses following allograft transplantation by administering an inhibitor of epsilon protein kinases c (εPKC) following transplantation.

### PRIORITY

The present application claims priority to U.S. Provisional Application Ser. No. 60/927,580, filed on May 4, 2007, which is hereby incorporated by reference in its entirety.

### STATEMENT REGARDING FEDERALLY SPONSORED RESEARCH

This invention was made with the support of National Institutes of Health Grant HL52141. Accordingly, the federal government may retain certain rights in the invention.

### TECHNICAL FIELD

The present subject matter relates to compositions and methods using an epsilon protein kinase C (εPKC) inhibitor for suppressing inflammation associated with transplantation.

### BACKGROUND

Transplantation is currently the most effective treatment for end-stage organ failure. However, allograft survival is affected by acute and chronic rejections due to incomplete histocompatibility, which causes severe inflammatory responses in transplant recipients (Thom, T. et al. (2006) Circulation. 113:e85-151; Taylor, D. O. et al. (2003) J. Heart Lung Transplant. 22:616-24.) While the survival rate of transplant patients continues to increase, largely due to the wide availability of immunomodulatory drugs, chronic or late-stage allograft rejection still causes high mortality. In addition, immunomodulatory drugs have significant side-effects.

Ten protein kinase C (PKC) isozymes are encoded in the human genome and each isozyme mediates distinct roles in normal and disease states (Nishizuka, Y. (1995) Faseb J. 9: 484-96). Using pharmaceutical agents selective for particular PKC isozymes, it was shown that δPKC and εPKC isozymes have opposing roles in cardiac ischemia and reperfusion (Chen, L. et al. (2001) Proc. Natl. Acad. Sci. U.S.A. 98:11114-9; Inagaki. K. et al. (2003) Circulation 108:2304-7; Inagaki, K. et al. (2006) Cardiovasc. Res. 70:222-30). In addition, using both knockout mice and PKC peptide modulators, Levine and collaborators demonstrated that εPKC inhibition profoundly suppressed acute and chronic inflammatory pain response (Hucho, T. B. et al. (2005) J. Neurosci. 25:6119-26) and Aksoy et al. suggested that εPKC controls inflammation and immune-mediated disorders (Aksoy, E. et al. (2004) Int. J. Biochem. Cell. Biol. 36:183-8).

It would be desirable to further explore the role of εPKC on transplant inflammation and rejection and to determine whether the administration of εPKC offers beneficial therapeutic effect to transplant recipients.

### REFERENCES

Thom, T. et al. (2006) Circulation. 113:e85-151.
Taylor, D. O. et al. (2003) J Heart Lung Transplant. 22:616-24.
Tanaka, M. et al. (2004) Circulation 110:II194-9.
Tanaka, M. (2005) J. Thorac. Cardiovasc. Surg. 129:1160-7.
Nishizuka, Y. (1995) Faseb J. 9: 484-96.
Smith, B. L. and Mochly-Rosen, D. (1992) Biochem. Biophys. Res. Commun. 188:1235-40.
Souroujon, M. C. and Mochly-Rosen, D. (1998) Nat. Biotechnol. 16: 919-24.
Chen, L. et al. (2001) Proc. Natl. Acad. Sci. U.S.A. 98:11114-9.
Inagaki. K. et al. (2003) Circulation 108:2304-7.
Inagaki, K. et al. (2006) Cardiovasc. Res. 70:222-30.
Hucho, T. B. et al. (2005) J. Neurosci. 25:6119-26.
Aksoy, E. et al. (2004) Int. J. Biochem. Cell. Biol. 36:183-8.
Tanaka, M. et al. (2005) J. Heart Lung Transplant. 24:446-53.
Corry, R. J. et al. (1973) Transplantation 16:343-50.
Chen, L. et al. (2001) Chem. Biol. 8:1123-9.
Billingham, M. E. et al. (1990) J. Heart. Transplant. 9:587-93.
Tanaka, M. et al. (1986) Br. Heart J. 55:575-81.
Booz, G. W. et al. (1995) Cardiovasc. Res. 30:537-43.
Crews, G. M. et al. (2005) Transplant. Proc. 37:1926-8.
Aziz, T. M. et al. (2003) J. Heart Lung Transplant. 22:663-73.
Tojima, Y. et al. (2000) Nature 404:778-82.
Chen, L. Y. et al. (2005) J. Biol. Chem. 280:22497-501.
Inagaki, K. et al. (2005) Circulation 111:44-50.
Altschul et al. (1990) J. Mol. Biol. 215:403-410.
Altschul et al. (1997) Nucleic Acids Res. 25:3389-3402.
Chen, L. et al. (2001) Chem. Biol. 8:1123-9.
Current Protocols in Molecular Biology (Ausubel et al., eds.), John Wiley & Sons.
Dorn, G. W. et al. (1999) Proc. Natl. Acad. Sci. U.S.A. 96:12798-12803.
Dorn, G. W. et al. (1999) Proc. Natl. Acad. Sci. U.S.A. 96:12798-12803.
Freidinger, R. M. (2003) J. Med. Chem. 46:5553.
Gellman, S. H. (1998) Acc. Chem. Res. 31:173.
Goodman, M. et al. (1996) Pure Appl. Chem. 68:1303.
Hagihara et al. (1992) J. Am. Chem. Soc. 114:6568.
Hirschmann et al. (2000) J. Am. Chem. Soc. 122:11037.
Johnson, J. A. and Mochly-Rosen, D. (1995) Circ Res. 76:654-63.
Johnson, J. A. et al. (1996) Circ. Res. 79:1086.
Liron, T. et al. (2007) J. Molecular and Cellular Cardiology 42:835-841
Karlin and Altschul (1990) Proc. Natl. Acad. Sci. USA 87:2264-2268.
Karlin and Altschul (1993) Proc. Natl. Acad. Sci. USA 90:5873-5877.
Kulesza, A. et al. (2003) Org. Letts. 5:1163.
Martin, Robin, Protein Synthesis: Methods and Protocols, Humana Press, Totowa, N.J. (1998).
Mitchell et al. (2000) J. Peptide Res. 56:318-325.
Patani, G. A. and Lavoie, E. J. (1996) Chem. Rev. 96:3147-3176.
Patil et al. (2003) J. Org. Chem. 68:7274-7280.
Ripka, A. S. and Rich, D. H. (1998) Curr. Opin. Chem. Biol. 2:441.
Rolhbard et al. (2000) Nature Med. 6:1253-1257.
Ron, D. and Mochly-Rosen, D. (1995) Proc. Natl. Acad. Sci. U.S.A. 92:492-496;
Ron, D. et al. (1994) Proc. Natl. Acad. Sci. USA 91:839-843.
Sambrook et al., Molecular Cloning: A Laboratory Manual, Cold Springs Harbor laboratory, 2nd ed., Cold Springs Harbor, N.Y. (1989).
Schechtman, D. et al. (2004) J. Biol. Chem. 279:15831-15840.
Scott et al. (2004) Org. Letts. 6:1629-1632.
Souroujon, M. C. and Mochly-Rosen, D. (1998) Nature Biotech. 16:919-924.
Theodore, L. et al. (1995) J. Neurosci. 15:7158-7167.
U.S. Pat. No. 5,804,604.
U.S. Publication Nos. US2004-0009919A1, US2005-0209160A1, US2005-0164947A1, and US2006-0148700A1.
Vives et al. (1997) J. Biol. Chem. 272:16010-17.
Williams, Paul Lloyd, et al., (1997) Chemical Approaches to the Synthesis of Peptides and Proteins, CRC Press, Boca Raton, Fla.
Wootton and Federhen (1993) Computers and Chemistry 17:149-163.
Zega and Urleb (2002) Acta Chim. Slov. 49:649-662.

### SUMMARY

In one aspect, a method for attenuating an inflammatory response due to allograft transplantation is provided, comprising administering an inhibitor of epsilon protein kinase C (εPKC).

In some embodiments, the inhibitor is administered after allograft transplantation.

In some embodiments, the inhibitor consists of between about 5-30 contiguous amino acid residues from a conserved set of amino acid resides from the sequence identified herein as εPKC.

In particular embodiments, the inhibitor is (i) EAVSLKPT (SEQ ID NO: 1) or (ii) a peptide having one or two conservative modifications to EAVSLKPT (SEQ ID NO: 1). In other embodiments, the inhibitor is β′-COP 285-292 (NNVALGYD; SEQ ID NO: 2). In yet another embodiment, the inhibitor is a mutated ψε-RACK peptide having, e.g., the amino acid sequence HNAPIGYD (SEQ ID NO: 3).

In some embodiments, the inhibitor is modified with a terminal amino acid residue that provides a reactive moiety. In particular embodiments, the inhibitor is modified with a cysteine residue.

In some embodiments, the reactive moiety is attached to a delivery peptide having activity to facilitate intracellular delivery of the inhibitor. In particular embodiments, the delivery peptide is selected from polyarginine, Drosophila Antennapedia homeodomain-derived sequence (RQIKIWFQNRRMKWKK; SEQ ID NO: 4), and Transactivating Regulatory Protein (Tat)-derived transport polypeptide (YGRKKRRQRRR; SEQ ID NO: 5) from the Human Immunodeficiency virus.

In some embodiments, the allograft transplantation involve the heart. In other embodiments, the allograft transplantation involves a kidney, gall bladder, lung, liver, eye, blood, bone marrow, or vessel.

In another aspect, a method for enhancing survival of an allograft transplantation recipient is provided, comprising

administering an inhibitor of εPKC.

In a further aspect, a kit comprising an inhibitor of εPKC and instructions for use in administration to an allograft transplantation recipient is provided. In some embodiments, the kit further comprises a delivery element for parenteral administration of the inhibitor.

### BRIEF DESCRIPTION OF THE DRAWINGS

FIG. 1 shows the results of an experiment performed in support of the present methods. A timeline of events is shown above the graph. The beating score in the days following transplantation are shown in the graph. Animals were treated with TAT₄₇₋₅₇-εV1-2 (n=9; solid line) or TAT₄₇₋₅₇ alone as a control (n=8, dotted line; line graphs on the bottom).

FIG. 2 shows histological features of the transplanted murine cardiac allografts treated with TAT (A and C-E) or εV1-2 (B and C′-E′) based on hematoxylin and eosin (H&E) staining (A and B with enlarged picture in small panels) and 3-methyl-4-diazobenzene (DAB) staining for a macrophage peroxidase marker (F4/80; C and C′), a T-cell marker (CD3; D and D′), or a B-cell marker (CD45R/B220; E and E′).

FIGS. 3A and 3B show histological staining of murine cardiac allografts treated with TAT (A) or εV1-2 (B) stained with Elastica van Gieson (EVG; scale bar=50 μm). FIG. 3C is a bar graph showing the amount of luminal narrowing in cardiac allografts treated with TAT or εV1-2.

FIGS. 4A and 4B show histological staining of murine cardiac allografts treated with TAT (A) or εV1-2 (B) stained with Masson's trichrome to assess cardiac fibrosis (blue). Scale bar=1 mm. For quantitative analysis, the fibrotic area in left coronary artery area, right coronary artery area and anterior side of septum were measured, averaged and indicated as percent of the total area. FIG. 4C is a graph summarizing the observed luminal narrowing.

FIGS. 5A-5C are bar graphs showing the local levels of TGF-β (A), PDGF-BB (B), and MCP-1 (C) in TAT or εV1-2-treated animals, as determined by ELISA. FIG. 5D shows the results of western blot analysis using RAW264.7 cells treated with 10 ng/ml TGF-β and incubated for 15 min (D). To observe the effect of εPKC on IκB polyubiquitination, εV1-2 or TAT control peptide (and MG132) were added to culture 10 min prior to TGF-β treatment.

### DETAILED DESCRIPTION

### I. Overview

The present methods relate to the administration of a selective εPKC inhibitor to suppress inflammation associated with allograft transplantation. The methods are based on the observation that the selective inhibition of εPKC with a peptide inhibitor prolonged allograft survival and improved functional recovery in a murine cardiac transplantation model. Inhibition of εPKC markedly attenuated the inflammatory response, resulting in reduced infiltration of macrophages and T-cells and a decrease in the attachment of mononuclear inflammatory cells to the arterial wall. εPKC administration further caused a substantial reduction in the extent of luminal narrowing and fibrosis in the allograft, preserving cardiac tissue architecture following transplantation.

To better understand the mechanism by which εPKC inhibitors suppress inflammatory response, the levels of several inflammatory cytokines, TGF-β, PDGF, and MCP-1 were measured before and after allograft transplantation. As expected, the process of transplantation significantly increased cytokine expression in control animals. Cytokine expression was attenuated/suppressed by εV1-2 treatment. The increase in MCP-1 and TGF-β that resulted from transplantation was abolished by εPKC inhibitor treatment, with the levels reduced to those observed in naïve allografts. Treatment with an εPKC inhibitor also reduced the increased expression of PDGF associated with allograft transplantation, although it was not statistically significant.

Without being limited to a theory, it is believed that decreased MCP-1 levels may result from decreased macrophage invasion into the perivascular area, subsequently decreasing vasculitis. In support of this theory, macrophage and T-cell invasion of perivascular and parenchymal regions was drastically decreased 30 days after transplantation in εV1-2-treated animals. TGF-β is a stimulator of proliferation and differentiation for cardiac fibroblasts (Aziz, T. M. et al. (2003) J. Heart Lung Transplant. 22:663-73) and its induction correlated with the level of fibrosis observed by histological analysis. In addition to the role in fibrosis, a decrease in TGF-β levels was reported to associate with inhibition of chronic rejection in rat kidney grafts (Tojima, Y. et al. (2000) Nature 404:778-82) and TGF-β expression in human cardiac allografts correlates with impaired cardiac function (Chen, L. Y. et al. (2005) J. Biol. Chem. 280:22497-501).

Alternatively, the inhibition of cellular inflammatory responses, such as NF-κB activation and subsequent effects on cytokine expression, may be an important mechanism by which the εPKC inhibitor reduces inflammation in allograft transplantation. εPKC is reported to control NF-κB activation in human embryonic kidney cells and human peripheral blood monocytes (Booz, G. W. et al. (1995) Cardiovasc. Res. 30:537-43; Crews, G. M. et al. (2005) Transplant. Proc. 37:1926-8). These observations are consistent with reduced levels of IκB poly-ubiquitination following administration of a εPKC inhibitor (see below).

In a further alternative mechanism, the εPKC inhibitor may affect the Toll-like receptor 4 (TLR4) pathway, which drives the IL-12-dependent Th1 responses of the immune system (Aksoy, E. et al. (2004) Int. J. Biochem. Cell. Biol. 36:183-8). One skilled in the art will recognize that such mechanisms are not mutually excusive, and that several mechanisms may contribute to the effects observed with the present methods.

Previous studies showed that acute activation of εPKC prior to organ transplantation mimics ischemic preconditioning (Tanaka, M. et al. (2004) Circulation 110:II194-9; Tanaka, M. (2005) J. Thorac. Cardiovasc. Surg. 129:1160-7; Inagaki. K. et al. (2003) Circulation 108:2304-7; Inagaki, K. et al. (2006) Cardiovasc. Res. 70:222-30). The present methods are drawn to sustained delivery of a εPKC inhibitor following transplantation. Several different therapeutic windows are noted. For example, sustained administration of an εPKC inhibitor is effective in reducing inflammation when administration is initiated about three days following transplantation. While the exact number of days following transplantation is likely not critical, it is preferred that εPKC inhibitor administration be administered following transplantation, rather than before or during transplantation.

Moreover, the ability of sustained administration of an εPKC inhibitor to attenuate rejection following allograft transplantation may be distinct from the ability of an εPKC activator to improve cardiac function following a brief pretreatment of the organ In any case, the ability of the εPKC inhibitor to reduce inflammation when administered following transplantation was unexpected in view of previous results.

An additional benefit of the present εεPKC inhibitors is the absence of side-effects in other organs (i.e., endogenous organs in the recipient) observed both in the present methods and those previously reported (Inagaki, K. et al. (2005) Circulation 111:44-50).

Since coronary artery disease and immune rejection are the major and leading causes of cardiac allograft dysfunction (Taylor, D. O. et al. (2003) J Heart Lung Transplant. 22:616-24), the ability to inhibit parenchymal inflammation and vasculitis using an εεPKC inhibitor will increase the survival rate, average survival time, and quality of life for transplant recipients.

Moreover, since inflammation is generally associated with allograft transplantation, and not limited to cardiac transplantation, it is expected that εPKC inhibitors will be effective in suppressing inflammation associated with a wide variety of allograft transplantations, including but not limited to kidney, gall bladder, lung, liver, eye, blood, bone marrow, and other organ transplantation.

### II. εPKC Inhibitors

The present methods feature the administration of an antagonist/inhibitor of εPKC. As used herein, an antagonist or inhibitor of εPKC is a compound that reduces εPKC expression, activity, activation, or stability, or reduces the effects of εPKC expression or activity, in a mammalian subject. An inhibitor of εPKC may be a compound that inactivates εPKC to form inactive εPKC, prevents εPKC from performing its biological functions, or otherwise antagonizes the activity of εPKC. The antagonist/inhibitor may be a competitive, non-competitive, or uncompetitive inhibitor of εPKC. In some embodiments, the inhibitor is a selective peptide inhibitor of εPKC, as opposed to an inhibitor of multiple PKC isozymes (e.g., α, β, δ, etc.).

As known in the art, εPKC is a serine/threonine kinase and is involved in a myriad of cellular process, including regulation of various physiological functions, such as the activation of various biological systems, including the nervous, endocrine, and exocrine systems.

The polypeptide sequences of murine, rat, and human εPKC are reproduced, below. The present compositions and methods contemplate the use of any one of these polypeptides, chimeric/hybrid polypeptides including sequence from one or more of these polypeptides, and/or fragments, variants, and derivatives, thereof.

| εPKC (Mus musculus); | |
|---|---|
| gi:6755084; ACCESSION: NP_036234 XP_994572 | |
| XP_994601 XP_994628 (SEQ ID NO: 6): | |
| 1 | MVVFNGLLKI KICEAVSLKP TAWSLRHAVG PRPQTFLLDP YIALNVDDSR IGQTATKQKT |
| 61 | NSPAWHDEFV TDVCNGRKIE LAVFHDAPIG YDDFVANCTI QFEELLQNGS RHFEDWIDLE |
| 121 | PEGKVYVIID LSGSSGEAPK DNEERVFRER MRPRKRQGAV RRRVHQVNGH KFMATYLRQP |
| 181 | TYCSHCRDFI WGVIGKQGYQ CQVCTCVVHK RCHELIITKC AGLKKQETPD EVGSQRFSVN |
| 241 | MPHKFGIHNY KVPTFCDHCG SLLWGLLRQG LQCKVCKMNV HRRCETNVAP NCGVDARGIA |
| 301 | KVLADLGVTP DKITNSGQRR KKLAAGAESP QPASGNSPSE DDRSKSAPTS PCDQELKELE |
| 361 | NNIRKALSFD NRGEEHRASS ATDGQLASPG ENGEVRPGQA KRLGLDEFNF IKVLGKGSFG |
| 421 | KVMLAELKGK DEVYAVKVLK KDVILQDDDV DCTMTEKRIL ALARKHPYLT QLYCCFQTKD |
| 481 | RLFFVMEYVN GGDLMFQIQR SRKFDEPRSR FYAAEVTSAL MFLHQHGVIY RDLKLDNILL |
| 541 | DAEGHCKLAD FGMCKEGIMN GVTTTTFCGT PDYIAPEILQ ELEYGPSVDW WALGVLMYEM |
| 601 | MAGQPPFEAD NEDDLFESIL HDDVLYPVWL SKEAVSILKA FMTKNPHKRL GCVAAQNGED |
| 661 | AIKQHPFFKE IDWVLLEQKK IKPPFKPRIK TKRDVNNFDQ DFTREEPILT LVDEAIIKQI |
| 721 | NQEEFKGFSY FGEDLMP |
| εPKC (Rattus norvegicus); | |
| ACCESSION: NP_058867 XP_343013 (SEQ ID NO: 7): | |
| 1 | MVVFNGLLKI KICEAVSLKP TAWSLRHAVG PRPQTFLLDP YIALNVDDSR IGQTATKQKT |
| 61 | NSPAWHDEFV TDVCNGRKIE LAVFHDAPIG YDDFVANCTI QFEELLQNGS RHFEDWIDLE |
| 121 | PEGKVYVIID LSGSSGEAPK DNEERVFRER MRPRKRQGAV RRRVHQVNGH KFMATYLRQP |
| 181 | TYCSHCRDFI WGVIGKQGYQ CQVCTCVVHK RCHELIITKC AGLKKQETPD EVGSQRFSVN |
| 241 | MPHKFGIHNY KVPTFCDHCG SLLWGLLRQG LQCKVCKMNV HRRCETNVAP NCGVDARGIA |
| 301 | KVLADLGVTP DKITNSGQRR KKLAAGAESP QPASGNSPSE DDRSKSAPTS PCDQELKELE |
| 361 | NNIRKALSFD NRGEEHRASS STDGQLASPG ENGEVRQGQA KRLGLDEFNF IKVLGKGSFG |
| 421 | KVMLAELKGK DEVYAVKVLK KDVILQDDDV DCTMTEKRIL ALARKHPYLT QLYCCFQTKD |
| 481 | RLFFVMEYVN GGDLMFQIQR SRKFDEPRSG FYAAEVTSAL MFLHQHGVIY RDLKLDNILL |
| 541 | DAEGHSKLAD FGMCKEGILN GVTTTTFCGT PDYIAPEILQ ELEYGPSVDW WALGVLMYEM |
| 601 | MAGQPPFEAD NEDDLFESIL HDDVLYPVWL SKEAVSILKA FMTKNPHKRL GCVAAQNGED |
| 661 | AIKQHPFFKE IDWVLLEQKK MKPPFKPRIK TKRDVNNFDQ DFTREEPILT LVDEAIVKQI |
| 721 | NQEEFKGFSY FGEDLMP |
| εPKC (Homo sapiens); | |
| ACCESSION: NP_005391 (SEQ ID NO: 8): | |
| 1 | mvvfngllki kiceavslkp tawslrhavg prpqtflldp yialnvddsr igqtatkqkt |
| 61 | nspawhdefv tdvcngrkie lavfhdapig yddfvancti qfeellqngs rhfedwidle |
| 121 | pegrvyviid lsgssgeapk dneervfrer mrprkrqgav rrrvhqvngh kfmatylrqp |
| 181 | tycshcrdfi wgvigkqgyq cqvctcvvhk rcheliitkc aglkkqetpd qvgsqrfsvn |
| 241 | mphkfgihny kvptfcdhcg sllwgllrqg lqckvckmnv hrrcetnvap ncgvdargia |
| 301 | kvladlgvtp dkitnsgqrr kkliagaesp qpasgsspse edrsksapts pcdqeikele |
| 361 | nnirkalsfd nrgeehraas spdgqlmspq engevrqgqa krlgldefnf ikvlgkgsfg |
| 421 | kvmlaelkgk devyavkvlk kdvilqdddv dctmtekril alarkhpylt qlyccfqtkd |
| 481 | rlffvmeyvn ggdlmfqiqr srkfdeprsr fyaaevtsal mflhqhgviy rdlkldnill |
| 541 | daeghcklad fgmckegiln gvttttfcgt pdyiapeilq eleygpsvdw walgvlmyem |
| 601 | magqppfead neddlfesil hddvlypvwl skeavsilka fmtknphkrl qcvasqnged |
| 661 | aikqhpffke idwvlleqkk ikppfkprik tkrdvnnfdq dftreepvlt lvdeaivkqi |
| 721 | nqeefkgfsy fgedlmp |

The inhibitor may be a protein, or other organic or inorganic compound, including a peptidomimetic small-molecule.

Suitable small molecules that may act as an inhibitor of εPKC may be determined by methods known to the art. For example, such molecules may be identified by their ability to translocate εPKC to its subcellular location. Such assays may utilize, for example, fluorescently-labeled enzyme and fluorescent microscopy to determine whether a particular compound or agent may aid in the cellular translocation of εPKC. Such assays are described, for example, in Schechtman, D. et al. ((2004) J. Biol. Chem. 279:15831-15840) and include use of selected antibodies. Other assays to measure cellular translocation include Western blot analysis as described in Dorn, G. W. et al. ((1999) Proc. Natl. Acad. Sci. U.S.A. 96:12798-12803) and Johnson, J. A. and Mochly-Rosen, D. ((1995) Circ Res. 76:654-63).

In certain forms of the invention, a protein inhibitor of εPKC may be utilized. The protein inhibitor may be in the form of a peptide. Protein, peptide and polypeptide are used interchangeably herein and refer to a compound made up of a chain of amino acid monomers linked by peptide bonds. Unless otherwise stated, the individual sequence of the peptide is given in the order from the amino terminus to the carboxyl terminus. Typically, peptides of εPKC having activity as an inhibitor of the translocation of εPKC are preferred, and generally have between about 5-30 amino acid residues, more preferably between about 6-25 amino acid residues, and still more preferably between about 6-15, and even more preferably from 6-12 or 8-15 amino acid residues.

The inhibitor of εPKC may be obtained by methods known to the skilled artisan. For example, the protein inhibitor may be chemically synthesized using various solid phase synthetic technologies known to the art and as described in, for example, Williams, Paul Lloyd et al. ((1997) Chemical Approaches to the Synthesis of Peptides and Proteins, CRC Press, Boca Raton, Fla.).

Alternatively, the protein inhibitor may be produced by recombinant technology methods as known in the art and as described, for example, in Sambrook et al., Molecular Cloning: A Laboratory Manual, Cold Springs Harbor laboratory, 2nd ed., Cold Springs Harbor, N.Y. (1989); Martin, Robin, Protein Synthesis: Methods and Protocols, Humana Press, Totowa, N.J. (1998); and Current Protocols in Molecular Biology (Ausubel et al., eds.), John Wiley & Sons, which is regularly and periodically updated. For example, an expression vector may be used to produce the desired peptide inhibitor in an appropriate host cell and the product may then be isolated by known methods.

An exemplary εPKC inhibitor peptide is TAT₄₇₋₅₇-εV1-2, which contains amino acid residues 47-57 of the HIV TAT transactivator protein (SEQ ID NO: 5), which directs entry into cells, and amino acid residues 14-21 of εPKC (i.e., EAVSLKPT; (SEQ ID NO: 1). This εPKC inhibitor is described in Chen, L. et al. ((2001) Chem. Biol. 8:1123-9) and in U.S. Publication Nos. US2004-0009919A1, US2005-0209160A1, US2005-0164947A1, US2006-0148700A1, which further describe the characterization of εPKC agonists and antagonists and which are incorporated by reference herein. Other εPKC inhibitor peptides may be used, including but not limited to peptides containing conservative amino acid substitutions and peptides having similarity to εPKC RACK amino acid residues, as described, below.

Polypeptides may be encoded by an expression vector, which may include, for example, the nucleotide sequence encoding the desired peptide wherein the nucleotide sequence is operably linked to a promoter sequence. As defined herein, a nucleotide sequence is “operably linked” to another nucleotide sequence when it is placed in a functional relationship with another nucleotide sequence. For example, if a coding sequence is operably linked to a promoter sequence, this generally means that the promoter may promote transcription of the coding sequence. Operably linked means that the DNA sequences being linked are typically contiguous and, where necessary to join two protein coding regions, contiguous and in reading frame. However, since enhancers may function when separated from the promoter by several kilobases and intronic sequences may be of variable length, some nucleotide sequences may be operably linked but not contiguous. Additionally, as defined herein, a nucleotide sequence is intended to refer to a natural or synthetic linear and sequential array of nucleotides and/or nucleosides, and derivatives thereof. The terms “encoding” and “coding” refer to the process by which a nucleotide sequence, through the mechanisms of transcription and translation, provides the information to a cell from which a series of amino acids can be assembled into a specific amino acid sequence to produce a polypeptide.

The εPKC inhibitor peptide may be capable of preventing activation signaling proteins, such as εPKC, that are activated in vivo by binding to a cognate polypeptide such as a receptor protein (RACK). Regions of homology between the εPKC signaling peptide and its RACK are termed “pseudo-RACK” sequences (ψ-RACK; Ron, D. et al. (1994) Proc. Natl. Acad. Sci. USA 91:839-843; Ron, D. and Mochly-Rosen, D. (1995) Proc. Natl. Acad. Sci. U.S.A. 92:492-496; Dorn, G. W. et al. (1999) Proc. Natl. Acad. Sci. U.S.A. 96:12798-12803; and Souroujon, M. C. and Mochly-Rosen, D. (1998) Nature Biotech. 16:919-924) and typically have a sequence similar to the PKC-binding region of the corresponding RACK.

In εPKC, the sequence HDAPIGYD (εPKC 85-92; Genbank Accession No. NP_{—}058867; SEQ ID NO: 9), named ψεRACK, has 75% homology with a sequence in εRACK consisting of amino acids NNVALGYD (RACK 285-292; SEQ ID NO: 2). A peptide corresponding to the ψεRACK sequence functioned as a εPKC-selective agonist (Dorn, G. W. et al. (1999) Proc. Nat. Acad. Sci. U.S.A. 96:12798-12803), possibly by stabilizing the “open” form of εPKC. Mutating Asp-86 in the ωεRACK sequence of εPKC to an Asn (i.e., D→N) produced an enzyme that translocated more slowly than the wild-type enzyme, presumably due to increased intramolecular interaction between the εRACK and the mutated ψεRACK-binding site in εPKC, which stabilized the “closed form.” Accordingly, the mutated ωεRACK peptide having the amino acid sequence HNAPIGYD (SEQ ID NO: 3), functioned as a εPKC antatgonist/inhibitor (Schechtman, D. et al. (2004) J. Biol. Chem. 279:15831-15840; Liron, T. et al. (2007) J. Molecular and Cellular Cardiology 42:835-841). Other mutated ψεRACK are expected to function as εPKC antatgonists/inhibitors. In addition, the polypeptide β′-COP has an εPKC binding motif (i.e., NNVALGYD; SEQ ID NO: 2), which is expected to function as an antagonist/inhibitor of εPKC (Dorn et al. (1999) Proc. Natl. Acad. Sci., USA; Schechtman et al (2004) J. Biol. Chem.).

Also included within these definitions, and in the scope of the invention, are variants of the peptides which function in reducing injury to a transplanted organ or tissue, modulating the activity and/or production of mediators of inflammation as described herein or modulating a pro-apoptotic event, or a combination thereof as described herein.

The peptides may include natural amino acids, such as the L-amino acids or non-natural amino acids, such as D-amino acids. The amino acids in the peptide may be linked by peptide bonds or, in modified peptides described herein, by non-peptide bonds. A wide variety of modifications to the amide bonds which link amino acids may be made and are known in the art. Such modifications are discussed in general reviews, including in Freidinger, R. M. ((2003) J. Med. Chem. 46:5553) and Ripka, A. S. and Rich, D. H. ((1998) Curr. Opin. Chem. Biol. 2:441). These modifications are designed to improve the properties of the peptide in one of two ways: (a) increase the potency of the peptide by restricting conformational flexibility; (b) increase the half-life of the peptide by introducing non-degradable moieties to the peptide chain.

Examples of strategy (a) include the placement of additional alkyl groups on the nitrogen or alpha-carbon of the amide bond, such as the peptoid strategy of Zuckerman et al, and the alpha modifications of, for example Goodman, M. et al. ((1996) Pure Appl. Chem. 68:1303). The amide nitrogen and alpha carbon may be linked together to provide additional constraint (Scott et al. (2004) Org. Letts. 6:1629-1632).

Examples of strategy (b) include replacement of the amide bond by, for instance, a urea residue (Patil et al. (2003) J. Org. Chem. 68:7274-7280) or an aza-peptide link (Zega and Urleb (2002) Acta Chim. Slov. 49:649-662). Other examples such as introducing an additional carbon “beta peptides” (Gellman, S. H. (1998) Acc. Chem. Res. 31:173) or ethene unit (Hagihara et al. (1992) J. Am. Chem. Soc. 114:6568) to the chain, or the use of hydroxyethylene moieties (Patani, G. A. and Lavoie, E. J. (1996) Chem. Rev. 96:3147-3176) are also well known. One or more amino acids may be replaced by an isosteric moiety such as, for example, the pyrrolinones of Hirschmann et al. (2000) J. Am. Chem. Soc. 122:11037), or tetrahydropyrans (Kulesza, A. et al. (2003) Org. Letts. 5:1163).

The εPKC inhibitors may be based on any mammalian polypeptide or polynucleotide sequence, including human sequences. Skilled artisans will recognize that, through the process of mutation and/or evolution, polypeptides of different lengths and having different constituents, e.g., with amino acid insertions, substitutions, deletions, and the like, may arise that are related to, or sufficiently similar to, a sequence set forth herein by virtue of amino acid sequence homology and advantageous functionality as described herein.

The peptide inhibitors described herein also encompass amino acid sequences similar to the amino acid sequences set forth herein that have at least about 50% identity thereto and function in reducing inflammation associated with allograft transplantation, modulating the activity of mediators of inflammation as described herein, modulating a pro-apoptotic event, or a combination thereof. Preferably, the amino acid sequences of the peptide inhibitors encompassed in the invention have at least about 60% identity, further at least about 70% identity, preferably at least about 80% identity, more preferably at least about 90% identity, and further preferably at least about 95% identity to the amino acid sequences of, e.g., εV1-2, β′-COP 285-292, and/or mutated ωεRACK, as set forth herein. Exemplary levels of sequence homology are 90%, 91%, 92%, 93%, 94%, 95%, 96%, 97%, 98%, and even 99%.

Percent identity may be determined, for example, by comparing sequence information using the advanced BLAST computer program, including version 2.2.9, available from the National Institutes of Health. The BLAST program is based on the alignment method of Karlin and Altschul ((1990) Proc. Natl. Acad. Sci. USA 87:2264-2268) and as discussed in (Altschul et al. (1990) J. Mol. Biol. 215:403-410; Karlin and Altschul (1993) Proc. Natl. Acad. Sci. USA 90:5873-5877; and Altschul et al. (1997) Nucleic Acids Res. 25:3389-3402. Briefly, the BLAST program defines identity as the number of identical aligned symbols (i.e., nucleotides or amino acids), divided by the total number of symbols in the shorter of the two sequences. The program may be used to determine percent identity over the entire length of the proteins being compared. Default parameters are provided to optimize searches with short query sequences in, for example, blastp with the program. The program also allows use of an SEG filter to mask-off segments of the query sequences as determined by the SEG program of Wootton and Federhen ((1993) Computers and Chemistry 17:149-163).

Accordingly, fragments or derivatives of peptide agonists described herein may also be advantageously utilized that include amino acid sequences having the specified percent identities to the amino acid sequences described herein to reduce inflammation associated with allograft transplantation, to modulate the activity and/or production of mediators of inflammation as described herein, to modulate a pro-apoptotic event, or a combination thereof.

Conservative amino acid substitutions may be made in the amino acid sequences described herein to obtain derivatives of the peptides that may advantageously be utilized in the present invention. Conservative amino acid substitutions, as known in the art and as referred to herein, involve substituting amino acids in a protein with amino acids having similar side chains in terms of, for example, structure, size and/or chemical properties. For example, the amino acids within each of the following groups may be interchanged with other amino acids in the same group: amino acids having aliphatic side chains, including glycine, alanine, valine, leucine and isoleucine; amino acids having non-aromatic, hydroxyl-containing side chains, such as serine and threonine; amino acids having acidic side chains, such as aspartic acid and glutamic acid; amino acids having amide side chains, including glutamine and asparagine; basic amino acids, including lysine, arginine and histidine; amino acids having aromatic ring side chains, including phenylalanine, tyrosine and tryptophan; and amino acids having sulfur-containing side chains, including cysteine and methionine. Additionally, amino acids having acidic side chains, such as aspartic acid and glutamic acid, are considered interchangeable herein with amino acids having amide side chains, such as asparagine and glutamine.

The derivatives include amino acid sequences where a given amino acid of one group (such as a non-polar amino acid, an uncharged polar amino acid, a charged polar amino acidic amino acid or a charged polar basic amino acid) is substituted with another amino acid from the same amino acid group. For example, it is know that the uncharged polar amino acid serine may be commonly substituted with the uncharged polar amino acid threonine in a peptide without substantially altering the functionality of the peptide. If one is unsure whether a given substitution will affect the functionality of the peptide, then this may be determined without undue experimentation using synthetic techniques and screening assays known in the art.

The εPKC inhibitor peptides described herein may be modified by being part of a fusion protein. The fusion protein may include a protein or peptide that functions to increase the cellular uptake of the peptide inhibitors, has another desired biological effect, such as a therapeutic effect, or may have both of these functions. For example, it may be desirable to conjugate, or otherwise attach, the εV1-2 peptide, an inhibitor derived from the ψεRACK peptide, or other εPKC inhibitor peptide, to a cytokine or other peptide that elicits a desired biological response. The fusion protein may be produced by methods known to the skilled artisan. The inhibitor peptide may be bound, or otherwise conjugated, to another peptide in a variety of ways known to the art. For example, inhibitor peptide may be bound to a carrier peptide or other peptide described herein by cross-linking wherein both peptides of the fusion protein retain their activity. As a further example, the inhibitor peptide may be linked or otherwise conjugated to each other by an amide bond from the C-terminal of one peptide to the N-terminal of the other peptide. The linkage between the transmembrane carrier or therapeutic peptide may be non-cleavable, with a peptide bond, or cleavable with, for example, an ester or other cleavable bond.

Furthermore, in other forms of the invention, the carrier protein or peptide that may increase cellular uptake of the peptide agonist or inhibitor may be, for example, a Drosophila melanogaster Antennapedia homeodomain-derived sequence (unmodified sequence may be found in Genbank Accession No. AAD19795; RQIKIWFQNRRMKWKK; SEQ ID NO: 4), and may be attached to the agonist or inhibitor by cross-linking via an N-terminal Cys-Cys bond as discussed in (Theodore, L. et al. (1995) J. Neurosci. 15:7158-7167; Johnson, J. A. et al. (1996) Circ. Res. 79:1086). The sequence may also be sought from Drosophila hydei and Drosophila virilis. Alternatively, the εPKC inhibitor may be modified by a Transactivating Regulatory Protein (Tat)-derived transport polypeptide (such as from amino acids 47-57 of Tat (i.e., YGRKKRRQRRR; SEQ ID NO: 5) from the Human Immunodeficiency Virus, Type 1, as described in Vives et al. ((1997) J. Biol. Chem. 272:16010-17); U.S. Pat. No. 5,804,604; as seen in Genbank Accession No. AAT48070; or with polyarginine, as described in Mitchell et al. ((2000) J. Peptide Res. 56:318-325) and Rothbard et al. ((2000) Nature Med. 6:1253-1257). The inhibitors may be modified by other methods known to the skilled artisan in order to increase the cellular uptake of the inhibitors.

### III. Supporting Studies

In studies performed in support of the present methods, hearts of FVB mice (H-2^{q}) were transplanted into C57BL/6 mice (H-2^{b}) to provide an animal model for allograft transplantation. Following transplantation, an isozyme-specific εPKC inhibitor was administered to the transplant recipients. The particular inhibitor was TAT₄₇₋₅₇-εV1-2 (i.e., εV1-2), which is described herein.

A. εPKC Inhibition Prolongs Allograft Survival

The εPKC-specific inhibitor, εV1-2, or the carrier/control peptide, TAT, was administered to the recipient animals using osmotic pumps to provide continuous treatment from day 3 following allograft transplantation until sacrifice on day 30. All animals were given 20 mg/kg/day of cyclosporine A (CyA) to ensure survival.

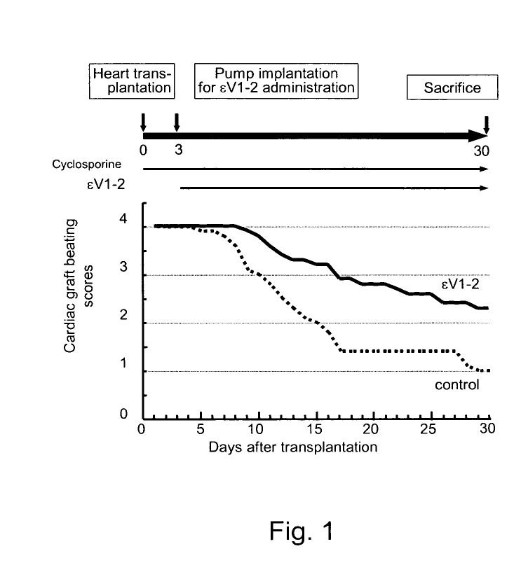

Animals treated with εV1-2-did not demonstrate toxic effects when compared to the TAT-treated group. Importantly, εV1-2 treatment significantly improved the beating score of cardiac allografts compared to TAT-peptide treatment (FIG. 1), suggesting that εV1-2 treatment augmented graft survival promoted by CyA without toxic side effects. εPKC inhibitor-treated animals had a similar beat score at 30 days as TAT-treated animals at 14 days (FIG. 1). Both groups showed normal behavior with no difference in body weight (TAT: 21.6±0.5 g; εV1-2; 21.7±0.9 g).

P values <0.05 were calculated for the beat scores on days 12-30. P=0.007 at day 15 and P<0.0001 at day 30. The overall p-value with repeated Anova test was <0.0001.

B. εPKC Inhibition Reduces Macrophages and T Cells Infiltration

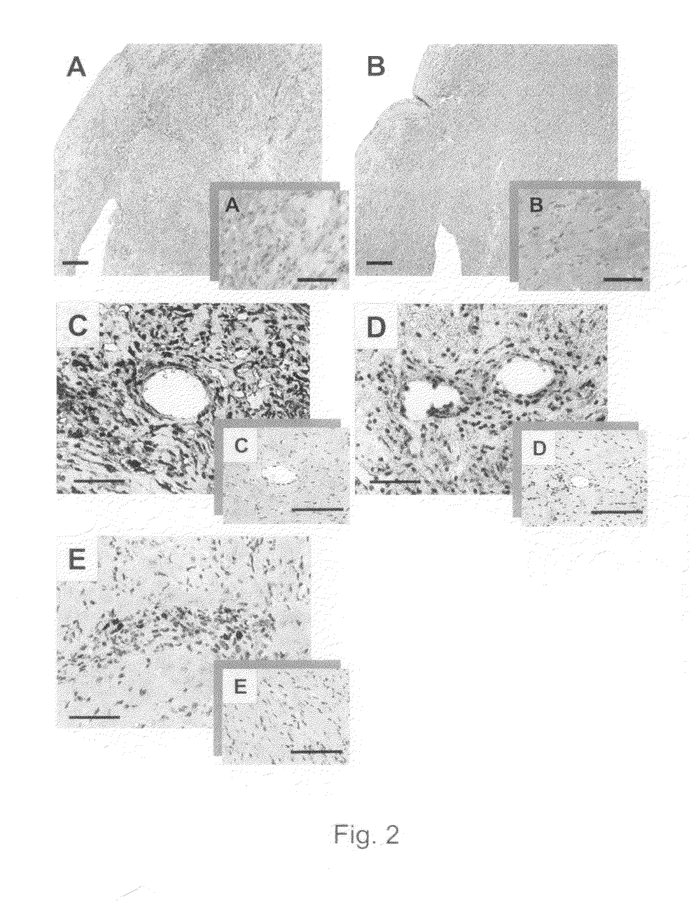

FIG. 2 shows histological features of the allografts in recipient animals treated with TAT (FIGS. 2A and 2C-E) or εV1-2 (FIGS. 2B and 2C′-E′) based on H&E staining for general tissue morphology; DAB staining for the macrophage marker, F4/80; antibody staining for the T-cell marker, CD3; or antibody staining of the B-cell marker, CD45R/B220. Diffused infiltration of inflammatory cells with severe multifocal myocyte damage, hemorrhage, and vasculitis were observed in control-treated (TAT) transplanted hearts (FIGS. 2A and 2C-E). In contrast, εV1-2 treatment markedly suppressed the inflammatory response in the transplant recipients (FIGS. 2B and 2C′-E′). Allografts from the εV1-2-treated animals showed only a few foci of perivascular mononuclear cell infiltratration. Parenchymal rejection (PR) scores were significantly lower in allografts treated with εV1-2 (2.3±0.3) compared to control animals (4.0±0.0; p<0.0001). Immunohistochemical analysis of tissue samples revealed that the infiltrating inflammatory cells were mainly macrophages and T-cells but not B-cells (FIGS. 2C-D).

Parenchymal rejection scores were significantly lower in allografts treated with εV1-2 (2.3±0.3) as compared to control (4.0±0.0; provide p<0.0001). Immunohistochemical analysis revealed those inflammatory cells were mainly macrophages and T cells but not B cells (FIG. 2C-G).

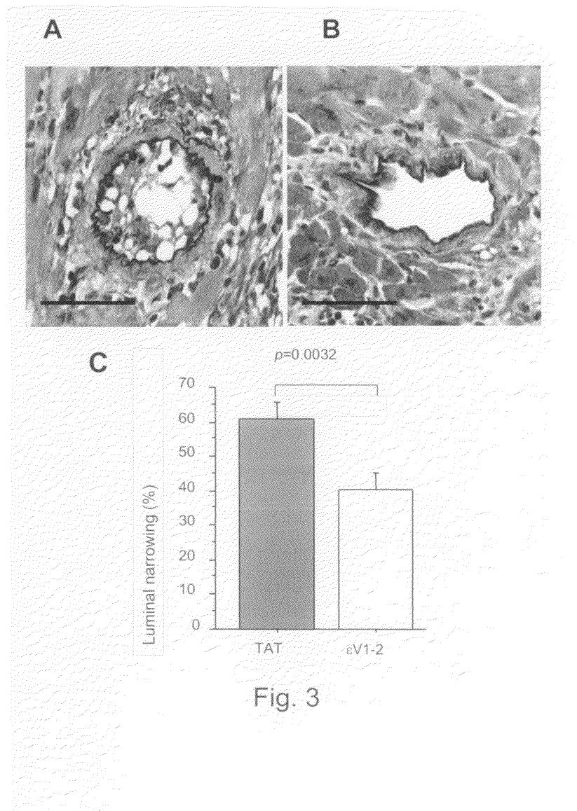

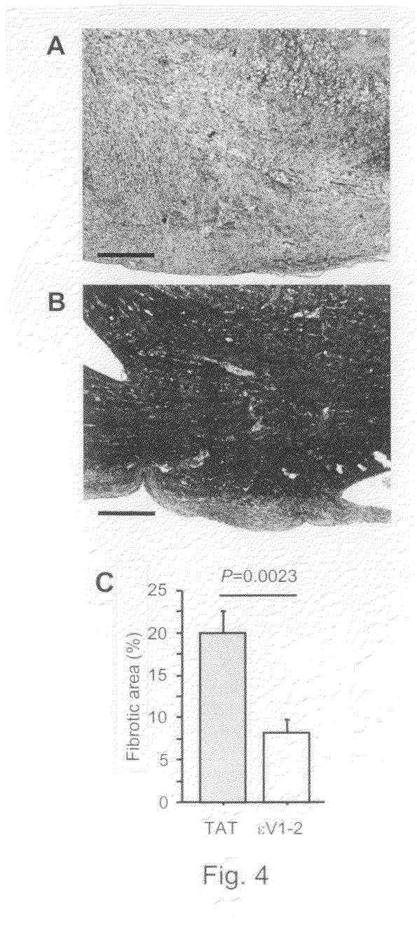

EVG staining to identify vascular structures demonstrated a significant decrease in the attachment of mononuclear inflammatory cells to the arterial walls in the allografts in the εV1-2-treated animals compared to the TAT-treated animals (FIGS. 3A and B). Luminal narrowing in middle-sized arteries was decreased by 30% in the εV1-2-treated animals compared to control animals (FIG. 4C). Furthermore, fibrosis, measured by Masson's Trichrome staining, was significantly decreased (40% of control) following εV1-2 treatment (FIG. 4).

C. εPKC Inhibition Reduces TGF-1 and MCP-1 Levels and Inflammatory Response in Culture

Organ transplantation is known to be associated with prolonged changes in the levels of a number of cytokines and chemokines. In particular, heterotopic cardiac transplantation significantly increases the levels of transforming growth factor-β (TGF-β), pletelet derived growth factor (PDGF), and monocyte chemoattractant protein-1 (MCP-1).

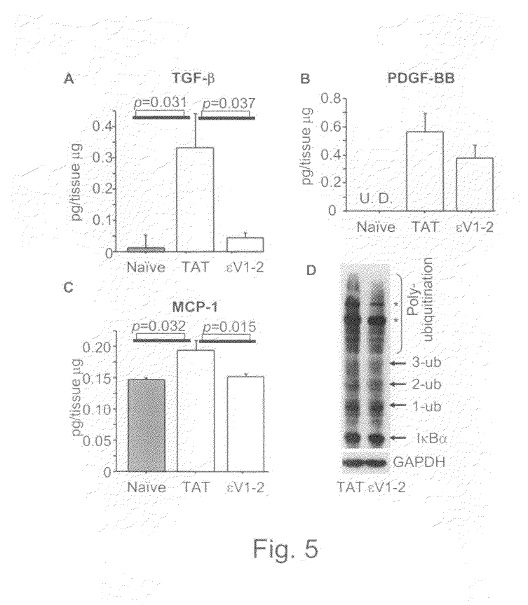

Administration of the εPKC inhibitor significantly reduced/suppressed the induction of TGF-β and MCP-1 (FIGS. 5A and C), demonstrating that the εPKC inhibitor suppressed the induction of inflammatory cytokines. However, administration of the εPKC inhibitor did not significantly suppress PDGF induction (FIG. 5B). Notably, the PDGF levels were below detection levels in untreated cardiac allografts.

Nuclear factor-κB (NF-κB) is one of the major transcription factors involved in inflammation. NF-κB is inhibited by inhibitors of NF-κB (IκB), which involves the ubiquitin-proteasome pathway. To determine whether the εPKC inhibitor affected the activation of NF-κB by εPKC, a macrophage derived cell line, RAW264.7, was treated with the inflammatory cytokine, tumor necrosis factor-α (TNF-α), in the presence of a proteasome inhibitor, MG132. The assay allowed measurement of poly-ubiquitination of IκB, while preventing the destruction (and inability to detect) poly-ubiquitinated IκB. As shown in FIG. 5D, the addition of the εPKC inhibitor, εV1-2, reduced induction of poly-ubiquitination of IκB by TNF-α to a greater extent than the TAT control peptide, indicating that NF-κB activity was down-regulated (i.e., reduced) by εV1-2 via poly-ubiquitination of IκB.

D. Summary of Results

Based on the results described herein, administration of an εPKC inhibitor following allograft transplantation significantly improved the beating score of a cardiac allograft throughout the treatment period. Infiltration of macrophages and T-cells into the allografts was reduced significantly and parenchymal fibrosis was decreased in animals treated with εV1-2 compared to control-treated animals. Finally, the increase in TGF-β and MCP-1 levels that accompanied allograft transplantation were almost abolished by εV1-2-administration. These data suggest that εPKC activity contributes to the chronic immune response in inflammation associated with allograft transplantation and that an εPKC-selective inhibitor, such as εV1-2, can augment current therapeutic strategies to suppress inflammation and prolong graft survival in humans.

All references cited herein are hereby incorporated by reference in their entirety. Modifications, substitutions, variations, and alternatives of the present inhibitors and methods will be apparent in view of the disclosure.

The following Examples are provide to illustrate the compositions and methods and are not intended to be limiting.

### EXAMPLES

### Example 1

### Animals

Male εVB (H-2^{q}) and C57BL/6J (H-2^{b}) mice, 6-8 weeks old, were purchased from Jackson Laboratory (Bar Harbor, Me.) and housed at the animal facility at Stanford University Medical Center (Stanford, Calif.). The εVB mice were used as allograft donors and the C57BL/6J mice were used as recipients. All mice were kept under standard temperature, humidity and timed lighting conditions and were provided mouse chow and water ad libitum. Animals were treated in compliance with the “Guide for the Care and Use of Laboratory Animals” prepared by the Institute of Laboratory Animal Resources, National Research Council, and published by the National Academy Press (revised 1996).

### Example 2

### Heterotopic Cardiac Transplantation

Heterotopic cardiac transplantation was performed according to the method of Corry et al. (Corry, R. J. et al., (1973) Transplantation 16:343-50) with some modifications. Anesthesia was induced with 5% inhaled isoflurane (Halocarbon Laboratories, River Edge, N.J.). During surgery, the animals were maintained on 2.5% inhaled isoflurane. Donor animals were systemically heparinized (50 mg/kg) before heart procurement. The donor heart was rapidly excised after coronary perfusion with ice-cold saline. The procured hearts were kept in ice-cold saline for 20 minutes. Because standard graft implantation averages 30 minutes, the total ischemic time was 50 minutes.

### Example 3

### Drug Administration

The selective εPKC inhibitor εV1-2 (εPKC amino acids 14-21; EAVSLKPT) was synthesized and conjugated to TAT (carrier peptide, amino acids 47-57; YGRKKRRQRRR) via a disulfide bond between cysteine residues added on amino termini of each peptides by American Peptides (Sunnyvale, Calif.), as previously described (Chen, L. et al. (2001) Chem. Biol. 8:1123-9). Recipient mice were treated with εV1-2 (n=9, 20 mg/kg/day), or with TAT as a control (13 mg/kg/day; n=8) using 0.1 mL osmotic pumps (release rate; 0.25 μL/hour, 30 mM of each peptide in sterile saline, Alzet, Alza) implanted subcutaneously from 3 to 30 days after transplantation. Recipients in both the εV1-2-treated group and control group received daily CyA (20 mg/kg/day) by intraperitoneal injection.

Allograft recipient mice were monitored daily. Graft viability was assessed by direct abdominal palpation of the heterotopically transplanted hearts and expressed as beating score, assessed by the Stanford cardiac surgery lab graft scoring system (Tanaka, M. et al. (2004) Circulation 110:II194-9).

### Example 4

### Histological Analysis

For histological analysis, grafts from 30-day post-transplantation animals (n=8 for TAT and n=9 for εV1-2) were harvested, fixed with 10% buffered formalin and embedded in paraffin. Sections were prepared from each specimen and stained with hematoxylin and eosin (H&E), Masson's trichrome, elastica von Gieson (EVG), or DAB for immunohisotochemistry by anti-F4/80, CD3 (Abcam) or CD45R/B220 (BD pharmingen). Parenchymal rejection (PR) severity was graded with a scale modified from the International Society for Heart and Lung Transplantation (Billingham, M. E. et al. (1990) J. Heart. Transplant. 9:587-93). The area of cardiac fibrosis were measured by a point-counting method (Tanaka, M. et al. (1986) Br. Heart J. 55:575-81). Percent luminal narrowing was calculated as [(inside area of internal elastic lamina)−(area of lumen)/(inside area of internal elastic lamina)]×100. Mid-sized coronary arteries (diameter; 40-120 μm) from multiple sections of the middle of the heart were analyzed (5 arteries for each graft) using ImageJ 1.35s software (Rasband, W. S., NIH, Bethesda, Md.).

### Example 5

### ELISA Determination of Local Cytokine Level

Harvested transplanted hearts were homogenized in 1× phosphate-buffered saline containing protease inhibitor cocktail (Sigma, St Louis, Mo.) and centrifuged at 30,000×g for 20 min at 4° C. The protein concentration of the supernatant was measured using Protein Assay (Bio-Rad, Hercules, Calif.) and 1-50 μg of protein were used for each analysis. All ELISA kits were purchased from R&D systems (Minneapolis, Minn.) and assays were performed according to the manufacturer's instructions.

### Example 6

### Western Blot Analysis

RAW264.7 cells were maintained in DMEM (Invitrogen) with supplemental 10% fetal bovine serum and penicillin-streptomycin solution (Invitrogen). Equal amounts of whole cell lysates were loaded to 10% SDS-polyacrylamide gel electrophoresis and were transferred to immobilon-P transfer membrane (Millipore). Anti-IκB (Cell Signaling) or GAPDH (Santa Cruz Biotechnology) antibodies were used for immunoblotting followed by HRP-conjugated anti-mouse (GE Healthcare) or rabbit (Santa Cruz Biotechnology) IgG antibodies.

### Example 7

### Statistical Analysis

Values were expressed as Mean±SEM. Statistical analysis was assessed by 1-way factorial ANOVA with Fisher's test, 2-way repeated ANOVA or Student's t-test when appropriate. A probability value <0.05 was considered significant.

What is claimed is:

1. A method for attenuating an inflammatory response due to allograft transplantation, comprising
administering an inhibitor of epsilon protein kinase c (εPKC).

2. The method of claim 1, wherein the inhibitor is administered after allograft transplantation.

3. The method of claim 1, wherein the inhibitor consists of between about 5-30 contiguous amino acid residues from a conserved set of amino acid resides from the sequence identified herein as εPKC.

4. The method of claim 1, wherein the inhibitor is (i) EAVSLKPT (SEQ ID NO: 1) or (ii) a peptide having one or two conservative modifications to EAVSLKPT.

5. The method of claim 1, wherein the inhibitor is modified with a terminal amino acid residue that provides a reactive moiety.

6. The method of claim 5, wherein the inhibitor is modified with a cysteine residue.

7. The method of claim 5, wherein the reactive moiety is attached to a delivery peptide having activity to facilitate intracellular delivery of the inhibitor.

8. The method of claim 7, wherein the delivery peptide is selected from polyarginine, Drosophila Antennapedia homeodomain-derived sequence (RQIKIWFQNRRMKWKK; SEQ ID NO: 4), and Transactivating Regulatory Protein (Tat)-derived transport polypeptide (YGRKKRRQRRR; SEQ ID NO: 5) from the Human Immunodeficiency virus.

9. The method of claim 1, wherein the allograft transplantation involves a kidney, gall bladder, lung, liver, eye, blood, bone marrow, or vessel.

10. A method for enhancing survival of an allograft transplantation recipient, comprising
administering an inhibitor of epsilon protein kinase c (εPKC).

11. A kit comprising an inhibitor of epsilon protein kinase c (εPKC) and instructions for use in administration to an allograft transplantation recipient.

12. The kit of claim 11, further comprising a delivery element for parenteral administration of the inhibitor.


SEQUENCE LISTING (9 sequences)

SEQ ID NO: 1 - protein, 8 aa, Artificial Sequence
  Synthetic peptide
EAVSLKPT

SEQ ID NO: 2 - protein, 8 aa, Artificial Sequence
  Synthetic peptide
NNVALGYD

SEQ ID NO: 3 - protein, 8 aa, Artificial Sequence
  Synthetic peptide
HNAPIGYD

SEQ ID NO: 4 - protein, 16 aa, Artificial Sequence
  Synthetic peptide
RQIKIWFQNRRMKWKK

SEQ ID NO: 5 - protein, 11 aa, Artificial Sequence
  Synthetic polypeptide
YGRKKRRQRRR

SEQ ID NO: 6 - protein, 737 aa, Mus musculus
MVVFNGLLKIKICEAVSLKPTAWSLRHAVGPRPQTFLLDPYIALNVDDSRIGQTATKQKTNSPAWHDEFVTDVCNGRKIELAVFHDAPIGYDDFVANCTIQFEELLQNGSRHFEDWIDLEPEGKVYVIIDLSGSSGEAPKDNEERVFRERMRPRKRQGAVRRRVHQVNGHKFMATYLRQPTYCSHCRDFIWGVIGKQGYQCQVCTCVVHKRCHELIITKCAGLKKQETPDEVGSQRFSVNMPHKFGIHNYKVPTFCDHCGSLLWGLLRQGLQCKVCKMNVHRRCETNVAPNCGVDARGIAKVLADLGVTPDKITNSGQRRKKLAAGAESPQPASGNSPSEDDRSKSAPTSPCDQELKELENNIRKALSFDNRGEEHRASSATDGQLASPGENGEVRPGQAKRLGLDEFNFIKVLGKGSFGKVMLAELKGKDEVYAVKVLKKDVILQDDDVDCTMTEKRILALARKHPYLTQLYCCFQTKDRLFFVMEYVNGGDLMFQIQRSRKFDEPRSRFYAAEVTSALMFLHQHGVIYRDLKLDNILLDAEGHCKLADFGMCKEGIMNGVTTTTFCGTPDYIAPEILQELEYGPSVDWWALGVLMYEMMAGQPPFEADNEDDLFESILHDDVLYPVWLSKEAVSILKAFMTKNPHKRLGCVAAQNGEDAIKQHPFFKEIDWVLLEQKKIKPPFKPRIKTKRDVNNFDQDFTREEPILTLVDEAIIKQINQEEFKGFSYFGEDLMP

SEQ ID NO: 7 - protein, 737 aa, Rattus norvegicus
MVVFNGLLKIKICEAVSLKPTAWSLRHAVGPRPQTFLLDPYIALNVDDSRIGQTATKQKTNSPAWHDEFVTDVCNGRKIELAVFHDAPIGYDDFVANCTIQFEELLQNGSRHFEDWIDLEPEGKVYVIIDLSGSSGEAPKDNEERVFRERMRPRKRQGAVRRRVHQVNGHKFMATYLRQPTYCSHCRDFIWGVIGKQGYQCQVCTCVVHKRCHELIITKCAGLKKQETPDEVGSQRFSVNMPHKFGIHNYKVPTFCDHCGSLLWGLLRQGLQCKVCKMNVHRRCETNVAPNCGVDARGIAKVLADLGVTPDKITNSGQRRKKLAAGAESPQPASGNSPSEDDRSKSAPTSPCDQELKELENNIRKALSFDNRGEEHRASSSTDGQLASPGENGEVRQGQAKRLGLDEFNFIKVLGKGSFGKVMLAELKGKDEVYAVKVLKKDVILQDDDVDCTMTEKRILALARKHPYLTQLYCCFQTKDRLFFVMEYVNGGDLMFQIQRSRKFDEPRSGFYAAEVTSALMFLHQHGVIYRDLKLDNILLDAEGHSKLADFGMCKEGILNGVTTTTFCGTPDYIAPEILQELEYGPSVDWWALGVLMYEMMAGQPPFEADNEDDLFESILHDDVLYPVWLSKEAVSILKAFMTKNPHKRLGCVAAQNGEDAIKQHPFFKEIDWVLLEQKKMKPPFKPRIKTKRDVNNFDQDFTREEPILTLVDEAIVKQINQEEFKGFSYFGEDLMP

SEQ ID NO: 8 - protein, 737 aa, Homo sapiens
MVVFNGLLKIKICEAVSLKPTAWSLRHAVGPRPQTFLLDPYIALNVDDSRIGQTATKQKTNSPAWHDEFVTDVCNGRKIELAVFHDAPIGYDDFVANCTIQFEELLQNGSRHFEDWIDLEPEGRVYVIIDLSGSSGEAPKDNEERVFRERMRPRKRQGAVRRRVHQVNGHKFMATYLRQPTYCSHCRDFIWGVIGKQGYQCQVCTCVVHKRCHELIITKCAGLKKQETPDQVGSQRFSVNMPHKFGIHNYKVPTFCDHCGSLLWGLLRQGLQCKVCKMNVHRRCETNVAPNCGVDARGIAKVLADLGVTPDKITNSGQRRKKLIAGAESPQPASGSSPSEEDRSKSAPTSPCDQEIKELENNIRKALSFDNRGEEHRAASSPDGQLMSPGENGEVRQGQAKRLGLDEFNFIKVLGKGSFGKVMLAELKGKDEVYAVKVLKKDVILQDDDVDCTMTEKRILALARKHPYLTQLYCCFQTKDRLFFVMEYVNGGDLMFQIQRSRKFDEPRSRFYAAEVTSALMFLHQHGVIYRDLKLDNILLDAEGHCKLADFGMCKEGILNGVTTTTFCGTPDYIAPEILQELEYGPSVDWWALGVLMYEMMAGQPPFEADNEDDLFESILHDDVLYPVWLSKEAVSILKAFMTKNPHKRLGCVASQNGEDAIKQHPFFKEIDWVLLEQKKIKPPFKPRIKTKRDVNNFDQDFTREEPVLTLVDEAIVKQINQEEFKGFSYFGEDLMP

SEQ ID NO: 9 - protein, 8 aa, Artificial Sequence
  Synthetic peptide
HDAPIGYD